(19) United States
(12) Patent Application Publication
(10) Pub. No.: US 2014/0308250 A1
(43) Pub. Date: Oct. 16, 2014

(54) CELL PREPARATIONS DEPLETED OF TCR ALPHA/BETA

(71) Applicant: Miltenyi Biotec GmbH, Bergisch-Gladbach (DE)

(72) Inventors: Rupert HANDGRETINGER, Bergisch Gladbach (DE); Volker Huppert, Bergisch Gladbach (DE)

(21) Appl. No.: 14/028,462

(22) Filed: Sep. 16, 2013

Related U.S. Application Data
(63) Continuation-in-part of application No. PCT/EP2012/054805, filed on Mar. 19, 2012.

(30) Foreign Application Priority Data
Mar. 17, 2011 (DE) 10 2011 001 380.6
Jun. 7, 2011 (EP) 11168949.3

(51) Int. Cl. C12N 5/078

(52) U.S. Cl. CPC C12N 5/0634
USPC 424/93.7; 435/325

(57) ABSTRACT

The invention relates to a composition, comprising a cell population that can be obtained from bone marrow or from blood, when the cell population is depleted of cells that express TCR alpha/beta and cells that express CD19. Such a pharmaceutical composition makes the reconstitution of the immune defense of a person as part of a bone marrow transplantation possible. By means of the invention, the time until the immune reconstitution is considerably shortened and the immune response after treatment are considerably reduced, in particular the occurrence of GvHD. The survival rate of the patient is considerably increased.

### CROSS-REFERENCE TO RELATED APPLICATION

This application claims priority to European Application No. EP11168949.3 filed Sep. 17, 2012, incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 212302002300 SeqList.txt, date recorded: Sep. 16, 2013, size: 37.4 KB).

### FIELD OF THE INVENTION

The present invention refers to TCR alpha/beta (TCR α/β)-depleted cell preparation, as well as their production and use for reconstituting of bone marrow and/or the immune system, in particular with respect to stem cell transplantation and with respect to the treatment of different types of cancer, such as leukemia.

### BACKGROUND OF THE INVENTION

More than 31,000 thousand stem cell transplantations are conducted each year in Europe of which about 13,000 are allogenous and 18,000 are autologous (Baldomero H, Gratwohl M, Gratwohl A, Tichelli A, Niederwieser D, Madrigal A, Frauendorfer K. The EBMT activity survey 2009: trends over the past five years. Bone Marrow Transplant. 2011 Feb. 28). Stem cells transplantations gain more and more importance in the treatment of hematological, oncological, immunological and genetic diseases (Zintl et al., Correction of fatal genetic diseases using bone marrow transplantation. Kinderarztl Prax. 1991 January-February; 59(1-2):6-9; Zintl, Bone marrow transplantation in childhood. I. Kinderarztl Prax. 1988 June; 56(6):259-64; Down J D, Mauch P M. The effect of combining cyclophosphamide with total-body irradiation on donor bone marrow engraftment. Transplantation. 1991 June; 51(6):1309-11) and constitutes for many of these diseases the only long term healing possibility (Eyrich et al., A prospective comparison of immune reconstitution in pediatric recipients of positively selected CD34+ peripheral blood stem cells from unrelated donors vs recipients of unmanipulated bone marrow from related donors. Bone Marrow Transplant. 2003 August; 32(4):379-90).

The main complications of stem cell transplantations originate from the reaction of the transplants against the recipients (Graft-versus-host-disease, GvHD or GvHR) from an erroneous engraftment of the transplanted stem cells, from the toxicity of the conditioning and the infections under therapy due to a prolonged or incomplete immune reconstitution.

For allogeneic transplantations, the incidence of therapy-associated mortality could be decreased with reduced conditioning regimes. Through reduced post-transplant immune suppression, the immunological effect of the transplant against the malignant tissue is favored and potential chemotherapy-associated side-effects are reduced for the recipient. This transplant versus tumor effect is mediated in particular through T and NK cells of the donor. In spite of a reduced conditioning, tumor progression does not increase (Valcárcel et al., Conventional versus reduced-intensity conditioning regimen for allogeneic stem cell transplantation in patients with hematological malignancies. Eur J Haematol. 2005 February; 74(2):144-51; Strahm et al. Reduced intensity conditioning in unrelated donor transplantation for refractory cytopenia in childhood. Bone Marrow Transplant. 2007 August; 40(4): 329-33). In order to avoid increased mortality, the immune system needs to be reconstituted as quickly as possible after an allogeneic transplantation so as to gain control over the tumor and over infections. This needs to be balanced with the occurrence of GvHD, which is favored when the immune suppression is stopped too early, when too many alloreactive T cells are used or when the antigen difference is too high and which in effect causes mortality and morbidity.

### 1) Passive TCD (CD34 Enrichment)

So far, patients in need of a stem cell transplantation were treated with a cell preparation of CD34 positive cells (stem cells) as an appropriate comparative therapy. This way, T cells are being depleted that play a major role in GvHD. However, together with the T cells and also with the NK cells, essential factors (effector cell population) are being lost that play role in healing.

The term graft-versus-host reaction (GvHR; german: Transplantat-Wirt-Reaktion; English: Graft-versus-Host-Disease (GvHD)) refers to an immunological reaction that may occur following an allogeneic bone marrow or stem cell transplantation (Jacobsohn D A, Vogelsang G B: Acute graft versus host disease. Orphanet J Rare Dis. 2007 Sep. 4; 2:35).

In a GvHD, in particular the T lymphocytes of a donor that are present in the transplant react against the host organism. From an immunological point, this is a reaction of the graft lymphocytes to the unfamiliar antigens of the patient.

One can distinguish an acute GvHD (aGvHD) and a chronic GvHD (cGvHD), wherein the chronic form may occur a 100 days after transplantation from the acute GvHD or as a de novo GvHD.

According to the Seattle Scheme, the acute and the chronic form are divided into grades. GvHD manifests itself at the skin, intestine and liver through exanthema and blisters on the body surface, diarrhea, ileus and increasing concentrations of bilirubin. For aGvHD, a subdivision from grade 0 to grade IV is performed based on the sum of the areas of manifestation and the severity of the manifestation. In order to avoid an aGvHD, prophylactic measures can be taken. Among those is the administration of immune suppressants, such as methotrexate (MTX), cyclosporine A (CsA), cortical steroids or the combination of any of these medicaments. (Martino R, Romero P et al.: “Comparison of the classic Glucksberg criteria and the IBMTR Severity Index for grading acute graft-versus-host disease following HLA-identical sibling stem cell transplantation. International Bone Marrow Transplant Registry.” Bone Marrow Transplant 1999; 24(3): S. 283-287 and Wikipedia, Graft-versus-Host-Reaktion).

The risk of developing GvHD is closely dependent on compatibility, which is determined by the human leukocyte antigen (HLA). A particularly high risk for suffering from aGvHD is present for an unrelated foreign donor and a HLA-incompatible donation. With respect to allogeneic transplantations of HLA identical sibling donors, ca. 35-60% of the patients develop an acute GvHD of light to average severity in spite of optimal medical precautionary measures and in spite of the administration of immune suppressant medicaments; ca. 10% suffer from a severe controllable GvHD (Kanda Y, Chiba S: “Allogeneic hematopoietic stem cell transplantation from family members other than HLA-identical siblings over the last decade (1991-2000).” Blood 2003; 102(4): S. 1541-1547).

A considerable fraction of the patients (ca. 30-65%) develop chronic GvHD that constitutes one of the most common side-effects and causes of death after stem cell transplantation long term (Ringdén et al., The graft-versus-leukemia effect using matched unrelated donors is not superior to HLA-identical siblings for hematopoietic stem cell transplantation. Blood. 2009 Mar. 26; 113(13): 3110-8; Ratanatharathorn et al., Phase III study comparing methotrexate and tacrolimus (prograf, FK506) with methotrexate and cyclosporine for graft-versus-host disease prophylaxis after HLA-identical sibling bone marrow transplantation. Blood. 1998 Oct. 1; 9 2(7): 2303-14.), and negatively affects the quality of life and delays the return to the work place (Wong et al., Long-term recovery after hematopoietic cell transplantation: predictors of quality-of-life concerns. Blood. 2010 Mar. 25; 115(12):2508-19; Sutherland et al., Quality of life following bone marrow transplantation: a comparison of patient reports with population norms. Bone Marrow Transplant. 1997 June; 19(11): 1129-36).

In addition, not only the prophylaxis but also the treatment of GvHD requires the administration of immune suppressants that can cause severe side-effects, for example, cortical steroid side-effects like diabetes, avascular necrosis, Cushing's syndrome or CsA/Tacrolimus side-effects like kidney damage, high blood pressure, Paresthesia and common infections of all kinds (Wong et al., Long-term recovery after hematopoietic cell transplantation: predictors of quality-of-life concerns. Blood. 2010 Mar. 25; 115(12):2508-19; Sutherland et al., Quality of life following bone marrow transplantation: a comparison of patient reports with population norms. Bone Marrow Transplant. 1997 June; 19(11): 1129-36; Ferrara J L, Levine J E, Reddy P, Holler E. Graft-versus-host disease. Lancet. 2009 May 2; 373(9674):1550-61).

### Immune Reconstitution

Another disadvantage of the present treatment method is the delayed immune reconstitution, that is, the delayed reestablishment of a functional immune system or hematopoietic system in the transplanted patient. The immune system needs ca. one to two years for reconstitution.

During this period, an increased likelihood exists for the patient to suffer from life threatening infections, foremost viral, bacterial or yeast infections or to die (Handgretinger R, Klingebiel T, Lang P et al. Megadose transplantation of purified peripheral blood CD34(+) progenitor cells from HLA-mismatched parental donors in children. Bone Marrow Transplant 2001; 27:777-83 and Platzbecker U, Ehninger G, Bornhauser M. Allogeneic transplantation of CD34+ selected hematopoietic cells: clinical problems and current challenges. Leuk Lymphoma 2004; 45:447-53).

The reason for this lies on the fact that all T cells, NK cells and further accessory cell populations that can help with immune reconstitution or the reconstitution of the hematopoietic system are being lost with the enrichment of CD34 positive stem cells.

The regeneration of T cells after transplantation and thereby the immune reconstitution occurs by two paths. The so-called central path is thymus-dependent and requires an intact thymus. T cells that have recently left the thymus are indicators for the recovery of the immune system. The determination of the T cell receptor excision circle (TREC) and immature T cells with the surface antigen CD45RA are suitable for characterization. The peripheral path of T cell reconstitution is thymus-independent and very important, since many conditioning regimes negatively affect the thymus. The expansion of mature T lymphocytes that are being transferred with the transplant assures the reconstitution of the immune system.

CD34+ selected transplantations in which the T cells are not transferred to the patient, therefore, show a delayed beginning of the reconstitution of the immune system (Sutherland et al., Reconstitution of naïve T cells and type 1 function after autologous peripheral stem cell transplantation: impact on the relapse of original cancer. Transplantation. 2002; 73: 1336-9; Rutella et al, Immune reconstitution after autologous peripheral blood progenitor cell transplantation: effect of interleukin-15 on T-cell survival and effector functions. Exp Hematol. 2001; 29:1503-16; Heining et al., Lymphocyte reconstitution following allogeneic hematopoietic stem cell transplantation: a retrospective study including 148 patients. Bone Marrow Transplant. 2007; 39: 613-22). The small GvHD rate for these kinds of transplantations is traded with a strongly delayed immune reconstitution. Therefore, the present weakness is the delayed immune reconstitution; that is, the delayed reestablishment of a functional immune system, which is associated with an increased risk for potentially lethal infections.

### Relapse

A further disadvantage of the T cell depletion is the heightened risk of the underlying disease, which was the reason for the stem cell transplantation (usually a leukemia) in the first place, to re-occur more often after the CD34 stem cell transplantation (Horowitz M M, Gale R P, Sondel P M et al. Graft-versus leukemia reactions after bone marrow transplantation. Blood 1990; 75:555-62), and also due to the removal of NK cells (natural killer cells), which have an anti-leukemic effect (Ruggeri L, Mancusi A, Capanni M et al. Exploitation of alloreactive NK cells in adoptive immunotherapy of cancer, Curr Opin Immunol 2005; 17:211-7).

### 2) Active TCD (CD3 Depletion)

CD3-depleted cell preparations were recently used, in which the T cells were depleted; NK cells, monocytes, granulocytes and CD34 negative stem cell progenitor cells were still present in the transplant. The risk of GvHD and therapy-associated early mortality (early treatment related mortality, TRM) was reduced, but these cell preparations also did not lead to a measurable increase of the survival rate (Lee C K, DeMagalhaes-Silverman M et al.: “Donor T-lymphocyte infusion for unrelated allogeneic bone marrow transplantation with CD3+ T-cell-depleted graft.” Bone Marrow Transplant 2003; 31(2): S. 121-128 and Wikipedia, Graft-versus-Host-Reaktion) or an improved immune reconstitution.

All references cited herein, including patent applications and publications, are incorporated by reference in their entirety.

### SUMMARY OF THE INVENTION

In a first aspect, the invention refers to a composition, in particular a pharmaceutical composition, comprising a cell population derivable from bone marrow or from blood. According to the invention, this cell population is depleted of TCR (T cell receptor) alpha/beta positive cells. Therefore, T cells are to be found in this (pharmaceutical) composition that are TCR gamma/delta positive, but only very few or almost none of the cells or, in the best case, no cells are TCR alpha/beta positive.

The term depletion refers to the significant reduction of cells from a cell population. Depletion can refer to a decrease of a cell type (which is defined through the presence of, for example, a cell surface marker, such as TCR alpha/beta or CD19) by at least two logarithmic steps, preferably by at least three logarithmic steps, particularly preferred by at least 4 logarithmic steps (e.g., 4.6 logarithmic steps), most preferred by at least four to five logarithmic steps.

The removal according to logarithmic steps is as follows: 1 log=90% removal of the unwanted cells, 2 log=99%, 3 log=99.9% and 4 log=99.99%. Methods for calculating the separation performance are known to a person of skill in the art and described, for example, in Bosio et al., Isolation and Enrichment of Stem Cells, Advances in Biochemical Engineering and Biotechnology, Springer Verlag Berlin Heidelberg, 2009.

Such a depletion is performed using the cell surface marker TCR alpha/beta and optionally also using CD19. The depletion can be performed with any technique known in state of the art, e.g. panning, elutriation or magnetic cell separation. Preferred is a depletion using magnetic cell separation (e.g. CliniMACS, Miltenyi Biotec GmbH) due to the high depletion efficiency.

The cell population obtainable from blood is in particular a cell preparation obtained by leukocyte apheresis or bone marrow puncture. Preferably, the cell preparation is obtained from a healthy donor who was previously treated with stem cell mobilizing drugs.

Preferably, the cell population of this (pharmaceutical) composition is also depleted with respect to CD19-positive cells. This leads to a cell population without B cells. This eradicates a possible transmittal of the Epstein-Barr-Virus (EBV) to the patient who is receiving the pharmaceutical composition, and therefore, reduced or no immune suppressants need to be administered.

In a preferred embodiment of the pharmaceutical composition, the composition comprises further at least one pharmaceutically acceptable carrier or additive. Such carriers or additives are known to the person of skill in the art.

The pharmaceutical composition can be administered against cancer, such as, leukemia and other diseases, e.g. acute myeloid leukemia, acute lymphatic leukemia, agranulocytosis, B-thalassemia, inborn error (HHS) as well as against solid tumors (e.g. neuroblastoma, sarcoma etc.) for which an allogeneic transplantation is indicated or a therapeutic effect of TCR alpha/beta depleted cell preparations is to be expected.

Moreover, a sufficient amount of CD34+ cells need to be transferred (at least two to four million per kg of body weight of the recipient) during an allogeneic transplantation in order to achieve a good reconstitution of the hematopoietic system and at least 25,000 TCR alpha/beta positive T cells per kg of body weight of the recipient should be administered to forgo or to dispense with immune suppression. B cells that are removed from the transplant to a CD19 depletion should be present in the smallest number possible or should be removed later in the recipient through, for example, the administration of an anti-CD19 antibody in vivo when the risk of an EBV infection and the complications arising from that shall be diminished.

The amount to be administered to a human patient of the depleted cell population lays typically between 2×10E10 bis 1×10E11 lymphocytes.

In a further aspect, the invention refers to the use of a cell population derived from bone marrow for the production of a pharmaceutical composition, wherein the cell population is depleted of TCR alpha/beta positive cells.

In a further aspect, the invention refers to the use of the pharmaceutical composition for the reconstitution of the hematopoietic system of a human after stem cell and/or bone marrow transplantation. This reconstitution is markedly faster compared to the reconstitutions known so far (e.g. with native bone marrow or CD34 positive stem cells from bone marrow or blood or mobilized, processed blood after leukapheresis) and thus, leads to a decreased need for transfusions of blood components and the possibility of a complete abdication of or a reduction of immune suppressant medicaments leading to reduced side-effects, less infections and a reduced mortality risk of the transplant recipient.

In a further aspect, the invention refers to a method, in particular, an in vitro method for the preparation of a population of cells. The method comprises the following steps:
Provision of bone marrow or blood of a donor (that is of a population that comprises, amongst others, TCR alpha/beta positive and TCR gamma/delta positive cells) and
Depletion of TCR alpha/beta positive cells from the cell population.

Preferably, the depletion of TCR alpha/beta positive cells is performed using an antibody or antigen-binding fragment against TCR alpha/beta. On the basis of the protein or nucleotide sequences according to SEQ ID NOs 4 to 14 of the receptor TCR alpha/beta (see Table 1 and the sequence protocol), an antibody or antigen fragment, or a derivative or conjugate thereof against TCR alpha/beta can be produced and used for the depletion of the TCR alpha/beta positive cells.

In a preferred embodiment, the method further comprises the following step:
Depletion of CD19 positive cells from the cell population. This step can be performed prior to, after, or parallel with the depletion of the TCR alpha/beta positive cells from the cell population.

In the method, the depletion of CD19 positive cells can be performed using an antibody or an antigen-binding fragment against CD19. On the basis of the protein or nucleotide sequence (SEQ ID NOs 1 to 3) of the surface marker CD19 (see Table 1 and sequence protocol) an antibody or an antigen-binding fragment, a derivative or conjugate thereof against CD19 can be produced and used for the depletion of CD19 positive cells.

The state of art enables a person of skill in the art knowing the protein or nucleotide sequences of TCR alpha/beta and CD19 (known in the state of the art) to generate an antibody, an antigen-binding fragment, or a derivative or conjugate thereof with known methods (e.g. Köhler, G. & Milstein, C. (1975): Continuous cultures of fused cells secreting antibody of predefined specificity. In: Nature, 256, 495-497; Shirahata S, Katakura Y, Teruya K. (1998): Cell hybridization, hybridomas, and human hybridomas. In: Methods in cell biology, 57, S. 111-145; Cole S P, Campling B G, Atlaw T, Kozbor D, Roder J C. (1984): Human monoclonal antibodies. In: Molecular and cellular biochemistry, 62, S. 109-120).

TABLE 1
Designations and SEQ ID NOS of the human protein and nucleotide
sequences of the surface markers CD19 and the subunits of the
receptors TCR alpha/beta (TCRA/TRBC). For the receptor subunits
TCR beta (TRBC), two protein and cDNA sequences are given
each period. The first amino acid and SEQ ID NO 11, “E”
(refers to “GAG in SEQ ID 13) is located on a splicing site and
therefore, possibly variable. In the protein sequence SEQ ID 10 and
corresponding in the nucleotide sequence, the amino acid and
nucleotide are therefore not specified.

| SEQ ID NO. | Designation | Sequence Type |
|---|---|---|
| 1 | CD19_human | protein sequence |
| 2 | CD19_human cDNA | nucleotide sequence |
| 3 | CD19_human genomic sequence | nucleotide sequence |
| 4 | TCRA_human | protein sequence |
| 5 | TCRA_human CDS | nucleotide sequence |
| 6 | TCRA_human genomic sequence | nucleotide sequence |
| 7 | TRBC1_human | protein sequence |
| 8 | TRBC1_human CDS | nucleotide sequence |
| 9 | TRBC1_human genomic sequence | nucleotide sequence |
| 10 | TRBC2_human | protein sequence |
| 11 | TRBC2_human_2 | protein sequence |
| 12 | TRBC2_human_CDS | nucleotide sequence |
| 13 | TRBC2_human_CDS_2 | nucleotide sequence |
| 14 | TRBC2_human genomic sequence | nucleotide sequence |

In a further aspect, the invention refers to the use of a method described here in for the reconstitution of the immune system and hematopoietic system of a human in connection with a stem cell or bone marrow transplantation.

In a different aspect, the invention refers to the use of a cell population obtained from a bone marrow or blood, wherein the cell population is depleted from cells that express TCR alpha/beta, for the reconstitution of the immune system of a human in connection with a bone marrow transplantation. As described above, CD19 positive cells are also depleted for this use.

In a further aspect, the invention refers to the use either of an antibody or antigen-binding fragment against TCR alpha/beta only or TCR alpha/beta and an antibody or an antigen-binding fragment against CD19 for the production of a population of cells that are depleted of TCR alpha/beta and/or CD19.

In a further aspect, the invention refers to a kit for producing a population of cells that are depleted of TCR alpha/beta and/or CD19. Such a kit comprises and antibody or an antigen binding fragment thereof against TCR alpha/beta and/or an antibody against CD19 or an antigen-binding fragment thereof.

In a further aspect, the invention refers also to the use of the kit described for the production of a population of cells that are TCR alpha/beta negative and CE19 negative. The cell population can be available in vitro and intended for research purposes or be available as a pharmaceutical composition, optionally with a pharmaceutically acceptable carrier and/or an additive.

In one embodiment, the invention does not refer to methods for treatment of the human or animal body by surgery or therapy and diagnostic methods practiced on the human or animal body.

### DETAILED DESCRIPTION OF THE INVENTION

### Advantages of the Invention

In spite of the improvements reached during the last years, stem cell transplantations are still associated with an acutely increased morbidity and an initial transplantation-related mortality. The main complication of stem cell transplantation arises from rejection reaction (graft-versus-host diseases) and their therapy, the slow immune reconstitution and reconstitution of the hematopoietic system and the infections after transplant resulting therefrom as well as toxicity of the conditioning.

It is possible through the use of TCR alpha/beta depleted cells or TCR alpha/beta and CD19 depleted cells (depleted cell population) to manipulate the hematological stem cell transplant such that the main complications after stem cell transplantation, that is the graft-versus-host disease as well as the slow immune reconstitution after stem cell transplantation and the long lasting susceptibility to bacterial, viral and yeast infections with a potentially lethal result, are significantly reduced.

At the same time the graft-versus-tumor reactivity of the cell mixture remains the same; that is, no increased risk of relapse is to be expected for the patient. A further advantage is a more stable engraftment of the transplant.

Through the significant reduction of so-called main complications, it is possible to dramatically reduce or completely relinquish those medicaments that are usually used for the treatment of these complications and that can lead to severe side-effects (and incur high costs). Through the relinquishment of the immune suppressant medicaments for GvHD prophylaxis, side-effects like for example infections (sepsis, candidosis, herpes simplex, etc.), which result from the drug-induced suppression of the immune system, can be avoided.

As a consequence, the standard of life of the patient as well as the success of the therapy can be improved markedly and at the same time the cost for the treatment can be reduced. The medicaments referred to above are medicaments for prophylaxis of GvHD as, for example, mofetilmycophenolate.

The depleted cell population allows not only for the reduction of infections and of GvHD, but allows also the use of a reduced conditioning regime (RIC). Reduced conditioning regimes can reduce the incidence of therapy-associated mortality for allogeneic transplantations and can be administered when the immunological effect of the transplant against the malignant tissue is to be used. This transplant against tumor effect is affected in particular through T and NK cells of the donor and therefore, is not usable with CD34 enriched stem cell preparations.

In this respect, there exists a significant additional benefit compared to the appropriate comparable therapies, since a sustainable improvement that has not yet been achieved with the appropriate comparable therapies is reached with regard to the therapy relevant benefit, in particular the healing of the disease, a significant prolongation of the survival time, the long term absence of severe symptoms and extensive avoidance of severe side-effects.

The use of TCR alpha/beta depletion strategy results in an early immune reconstitution with values of more 100 CD4 cells/μl within six weeks after the transplantation, compared to 10 months, as reported by Aversa et al. (Treatment of high-risk acute leukemia with T-cell-depleted stem cells from related donors with one fully mismatched HLA haplotype. N Engl J Med. 1998 Oct. 22; 339(17):1186-93.).

### Technical Results

Depletion runs (Depletions) that are performed with CliniMACS TCRαβ-Biotin (Miltenyi Biotec GmbH) show that the efficiency of the depletion is very robust with an average log depletion of 4.6 (FIG. 1).

CD19 is a surface molecule on T cells. That term CD19 positive cells refers to cells to which a CD19 molecule, for example, an antibody can specifically bind to the CD19 molecule on the surface of the T cell.

TCR alpha-beta is a surface molecule on T cells. The term TCR alpha/beta positive cells refers to a cell to which a TCR alpha/beta-binding molecule, for example, an antibody can specifically bind to the TCR alpha/beta molecule on the surface of the T cell.

Antibody means a monoclonal, polyclonal antibody (Harlow and Lane, “Antibodies, A Laboratory Manual”, CSH Press, Cold Spring Harbor, USA) that binds to a molecule or a derivative of these antibodies that retains binding capacity or largely retains the binding capacity. Preferred derivatives of these antibodies are chimeric antibodies comprising, for example, chimeric antibodies of a variable region or the mouse or the rat and a human constant region. The term “antibody” comprises also bi-functional or bi-specific antibody and antibody constructs like Fvs (scFv) from single chain or antibody fusion proteins. The term “scFv” (single chain Fv Fragment) is known to a person of skill in the art and is preferred that the fragment is produced in a recombinant fashion.

The antibody can be human or humanized. The term “humanized antibody” means that at least one antibody binding site ((complementary determining region (CDR)), like for example, CDR3 and preferably all six CDRs were substituted by CDRs from a human antibody with the desired specificity. Optionally, the non-human constant region(s) was replaced by a constant region(s) of a human antibody. Methods for producing human antibodies are described for example in EP 0239400 A1 and WO 90/07861 A1.

The term antigen-binding fragment refers to a fragment of an antibody as defined above like for example separated light and heavy chains, Fab, Fab/c, Fv, Fab′ F(ab′)2. An antigen-binding fragment can comprise a variable region of the light chain and a variable region of the heavy chain, not necessarily both together.

### Clinical Results

11 patients were treated: Eight patients with a TCRαβ depleted transplant from a haploid donor and three patients with a transplant from a matched unrelated donor.

Due to the very robust depletion and therefore, the small number of cells of TCRαβ⁺ T cells in the transplant, no GvHD prophylaxis in the form of immune suppressant medicaments, like for example MMF or CsA, were needed to be admitted after the transplantation.

The graft-versus-Host disease was reduced in the treated patients (FIG. 2). In all cases, GvHD symptoms could only be seen on the skin and symptoms were only temporary. 36% of the patients showed GvHD stage I, 18% showed GvHD stage II. GvHD stage III was not observed. This is remarkable, since no GvHD prophylaxis in the form of immune suppressant medicaments was administered after the transplantation. In 10 out of 11 patients, the transplant became engrafted between day seven and day nine.

In all patients, a strongly accelerated immune reconstitution was observed. As show in FIG. 1, the immune reconstitution was markedly faster with TCRαβ/CD19 depleted cell populations than with CD3/CD19 depleted cells (transplants).

Shown is the immune reconstitution until >200 cells/μl are reached after the transplantation of TCRαβ/CD19 depleted peripheral blood stem cells (N=11) in comparison to CD34 enriched peripheral blood stem cells (historic control, N=13). The data regarding immune reconstitution after stem cell transplantation with CD34 enriched cells was taken from the following publication: Br J Haematol. 2001 August; 114(2):422-32.

In all of these patients, a very fast immune reconstitution was observed that was utterly surprising and so far cannot be explained, because the reconstitution is also markedly faster compared to the administration of un-manipulated bone marrow that contains all immune reconstituting cells of the donor and therefore, a faster immune reconstitution was to be expected with un-manipulated bone marrow than with the administration of TCR alpha/beta and CD19 depleted cell preparations, which contain significantly less immune reconstituting cells.

FIG. 5 shows the immune reconstitution of patients that received three successive stem cell transplantations. The first stem cell transplantation was from a MUD donor with un-manipulated bone marrow, the second from a haploid donor with CD3/CD19 depleted peripheral blood stem cells (PBSC). In both cases, no reconstitution of the immune system occurred. Only when the patient received a third stem cell transplantation with TCR alpha/beta and CD19 depleted PBSCs from the father, a very fast immune reconstitution occurred.

The GvHD was other than expected not increased in the cases of TCR alpha/beta and CD19 transplantations (FIG. 2). It needs to be borne in mind that only the skin was affected by GvHD and that the GvHD symptoms were only temporary, although no immune suppressants were give for treatment.

A reason for the fast immune reconstitution could be the TCR gamma/delta cells, which are present in a TCR alpha/beta depleted cell preparation in the transplant but are not present in a CD34 positive stem cell transplant.

### Stem Cell and Bone Marrow Transplantation

For a bone marrow transplantation, about one liter of a bone marrow-blood mixture is removed from the pelvic bone of the donor under general anesthesia.

In order to remove stem cells from the blood, the body's own hormone-like substance is administered to the donor over several days that stimulates the production of stem cells and their transfer from the bone marrow to the blood circulatory system. The methods for the pre-treatment of the donors for the removal of bone marrow or blood stem cells are state of the art and known to the skilled artisan.

### Procedure

The aim of the blood stem cell transplantation is to equip the recipient with a healthy stem cell population that can differentiate into blood cells. Thereby, the deficient or the pathological cells of the recipients are being replaced (Beers and Berkow 2000). In allogeneic transplantations, the tissue stems from a healthy donor. This can be an identical sibling twin, an HLA identical sibling, a non-HLA family member (mismatched related donor), a haploid identical donor or an unrelated HLA-compatible donor. The main target of the allogeneic transplantation is to substitute the ill or defective hematopoietic system, like for example the bone marrow of the recipient, completely by a healthy, functional hematopoietic system (comprising the immune system). The stem cell transplantation can, however, also be performed with autologous, that is, the patient's own cells.

### Donors (IdSib, MUD, Haploid Donors)

A donor of first choice is an identical sibling (Identical Sibling=IdSib) with respect to the relevant histocompatibility antigens HLA-A, B, C, DRB1 and DQB1. However, such an identical sibling can only be found in ca. 30% of the cases, such that often an HLA-identical unrelated donor (matched unrelated donor, MUD) needs to be found (Ottinger et al., 2001). Since far from all histocompatibility antigens are known and only a limited number of alleles can be tested, one needs to assume a worse match with an identical unrelated donor than with a sibling donor.

A remarkable segment of the patient population remains without donor. For these patients, related donors can be used that agree with a recipient only in one haplotype of there HLA allele, that is, haplo-identical.

Transplants of unrelated donors (MUD) are used most often for hematopoietic stem cell transplantations (Blood 2003; 101(4): 1630-6).

### MUD: Un-Manipulated Transplant

For un-manipulated transplants in the MUD setting, GvHD is the main complication. Severe cases of GvHD are to be regarded as life threatening and require massive therapy with immune suppressant substances for which response rates of ca. 40% have been described (Vogelsang et al., 2003). The acute GvHD stages II-IV: V:33%; C: 51% and stage III-IV: V:11.7%; C: 24.5% (Finke et al., Lancet, 2009).

Alternatively, CD34 enriched transplants were used in the MUD setting in order to reduce GvHD and to avoid side-effects that go along with the necessary GvHD prophylaxis. The disadvantage is the delayed immune reconstitution with all the consequences as already described.

### Actual Transplantaion

The actual transplantation can be divided into two phases. With the conditioning through chemo- and/or radiation therapy, the immune system of the recipient is destroyed so that the transferred or transplanted bone marrow or stem cells are not being rejected. That is to say, the recipient is being prepared for the engraftment of the transplant. The better this is achieved, the slower the risk of a non-engraftment or rejection of the transplant. Depending on the strength of the conditioning, the goal to be achieved is to destroy the remaining leukemic or malignant cells in the patient. The transplantation is performed in an intravenous manner at day 0. Until the engraftment of the transplant and the fading of the immediate toxicity, the patient remains usually in a ward suited for such a case. After the engraftment of the transplant and the waning of the immediate toxicity, a rigorous monitoring is necessary during the first three months. The intensity of the monitoring depends heavily on the type of the donor and the complications and merges into a regular life-long after care.

### Indications

All indications that require an allogeneic stem cell transplantation can be treated with the cell population or pharmaceutical composition of the invention. All severe inborn and acquired malignant and non-malignant diseases of hematopoietic system are generally indications for an allogeneic stem cell transplantation. Further indications are malignant diseases that respond to a dose-intensification of the chemotherapy or radiation therapy.

Immune suppressants like cyclosporine, corticosteroids, antimetabolites and monoclonal anti-lymphocytic-antibodies are used routinely nowadays in order to control GvHD better.

### Depletion of TCRα/β⁺ Cells

The depletion of TCRα/β⁺ is described for example in Chaleff et al., Cytotherapy, 2007, 9, 746-754 or as described in the respective protocol of Miltenyi Biotec GmbH.

Combined depletion of TCRα/β⁺/CD19⁺ Cells

The leukapheresis product is diluted with CliniMACS® PBS/EDTA Buffer (with HSA to a final concentration of 0.5% (w/v)) prior to magnetic labeling. The leukapheresis product is diluted up to the 3-fold volume of the leukapheresis product without exceeding the maximum volume of 600 ml.

The cells are centrifuged at 200×g for 15 minutes (min) at room temperature (+19° C. to +25° C.). The supernatant is discarded. The optimal weight for the labeling is 88 g (±5 g). The pellet is re-suspended and the weight is determined. The cells are labeled with CliniMACS® TCRα/β-Biotin and mit CliniMACS® CD19 Reagent, one vial (7.5 ml) of CliniMACS® TCRα/β-Biotin and one vial of (7.5 ml) CliniMACS® CD19. The vials are stored at +2° C. to +8° C. and processed cold. Cells and reagents are mixed; the incubation time is 30 min. at 25 rpm at room temperature (+19° C. to +25° C.).

Leukoapheresis product and buffer are mixed and stirred lightly, followed by centrifugation at 300×g for 15 min without break and room temperature (+19° C. to +25° C.). The supernatant is discarded. The pellet is re-suspended and washed. Buffer is added until the weight of ca. 190 g is reached for the magnetic labeling with the TCRα/β-Biotin labeled cells with the CliniMACS® Anti-Biotin reagents. The (7.5 ml) CliniMACS® Anti-Biotin reagents that were cooled at +2° C. to +8° C. are added to the cells, incubated for 30 min at light stirring of the cells at 35 rpm and room temperature (+19° C. to +25° C.).

Leukoapheris product and 500 ml of buffer are stirred lightly and mixed, subsequently centrifuged at 300×g for 15 min with break at room temperature (+19° C. to +25° C.). The supernatant is discarded; the pellet is re-suspended until 150 g are reached. It is recommended to adhere to a maximal concentration of 0.4×10⁹ cells per ml.

The program DEPLETION 3.1 is started on the CliniMACS®^{plus} instrument and the instructions given by the manufacturer Miltenyi Biotec GmbH are followed. After the automatic separation has ended, the cell concentration is determined. The cells are depleted of TCR alpha/beta and CD19 after the automatic separation by ca. three to five log steps. The obtained TCR alpha/beta and CD19 depleted cell preparation can be used for transplantation after it has been re-suspended in a solution suitable for the transplantation. A person of skill in the art knows such solutions.

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise. It is understood that aspects and variations of the invention described herein include “consisting” and/or “consisting essentially of” aspects and variations.

### BRIEF DESCRIPTION OF THE DRAWINGS

FIG. 1: Immune reconstitution after stem cell transplantation until >200 cells/μl have been reached.

Shown is the immune reconstitution until >200 cells/μl have been reached after transplantation of TCRαβ/CD19 depleted peripheral blood stem cells (N=11) in comparison to CD34 enriched peripheral blood stem cells (historical control, N=13). The data for immune reconstitution after stem cell transplantation with CD34 enriched cells were taken from the following publication: Br J Haematol. 2001; 114: 422-32.

FIG. 2: GvHD after stem cell transplantation

The incidence of acute GvHD in patients that have received TCRαβ/CD19-depleted haplo-identical transplants. Control groups (historical controls): Patients with CD34 enriched haplo-identical transplants and patients with un-manipulated bone marrow from identical unrelated donors and methotrexate/CsA for GvHD prophylaxis. The data of the control groups were taken from Lang et al. 2007, Zeitschrift für Regenerative Medizin, Nr. 1: 32-39.

FIG. 3A and FIG. 3B: Analysis of T cell receptor β repertoire diversity from the confirmation of the thymus dependent T cell reconstitution

In exemplary fashion, the result of the analysis of TCRβ repertoire diversity through CDR3 spectra typing is shown for the measurement of the thymus-dependent T cell reconstitution for a patient at day 12 (FIG. 3A) and day 33 (FIG. 3B). The T cell receptor CDR3 region is the only hyper-variable region that is not germline encoded. This TCRαβ region is generated in the thymus, partly through recombination. The method is described in Bone Marrow Transplant. 2008 October; 42 Suppl 2: S54-9.

FIG. 4: T cell receptor excision circles (TRECs) in the peripheral blood for the quantification of T cells stemming from the thymus

In an exemplary fashion, the result of the determination of T cell receptor excision circles (TRECs) in peripheral blood shown for the confirmation that T cells are produced in the thymus after transplantation. This method was used in many studies after the transplantation in order to assess the activity of the thymus. This method is described in Zhonghua Yi Xue Za Zhi 2007 Aug. 28; 87(32):2265-7.

FIG. 5A, FIG. 5B, and FIG. 5C: Clinical results with children. Comparative analysis of the T cell regeneration of a patient.

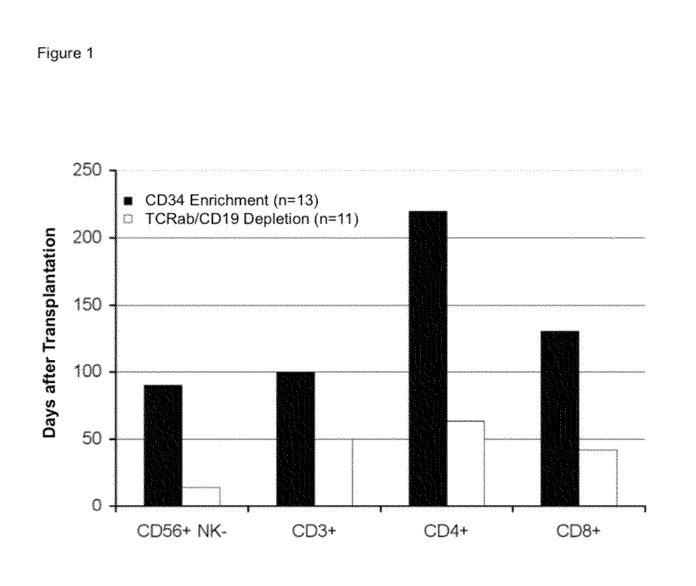

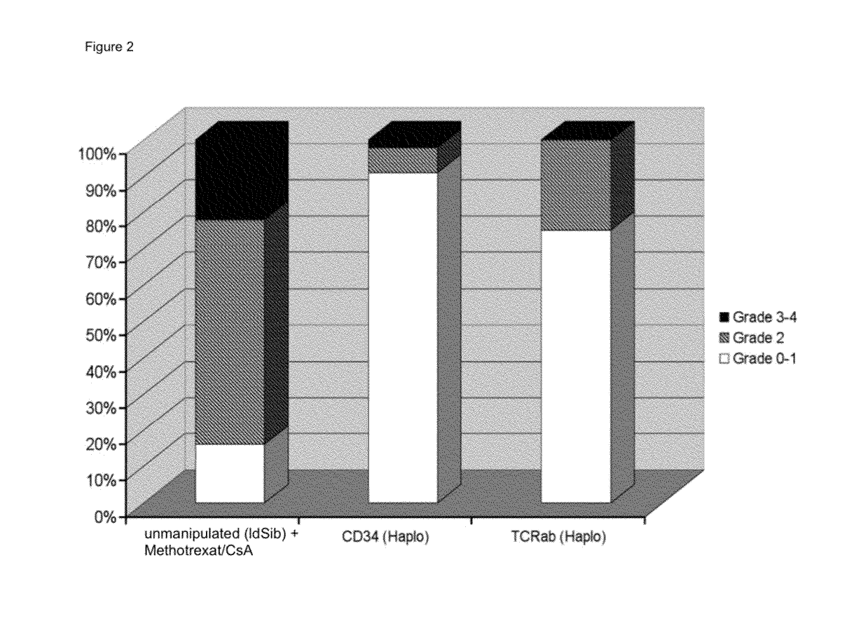

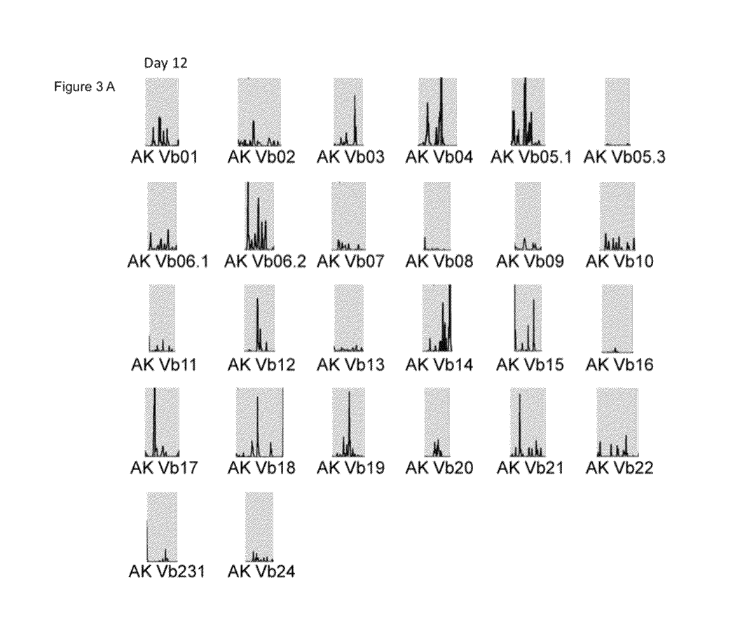

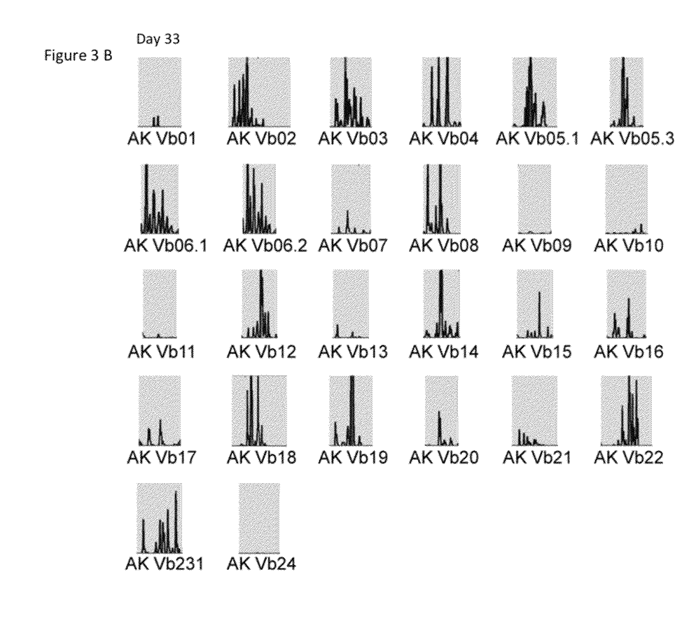

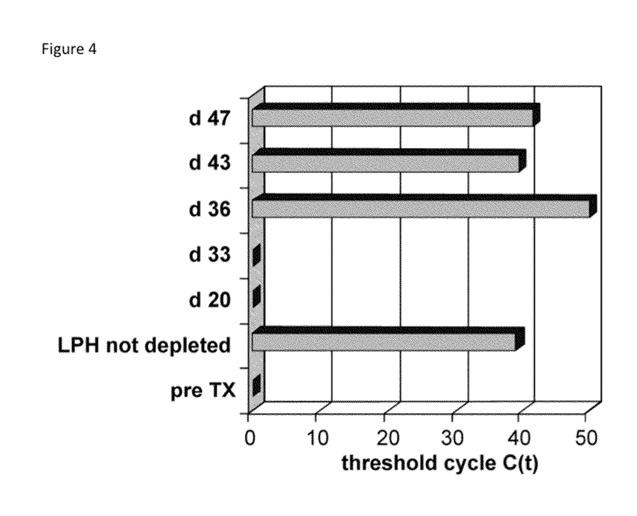

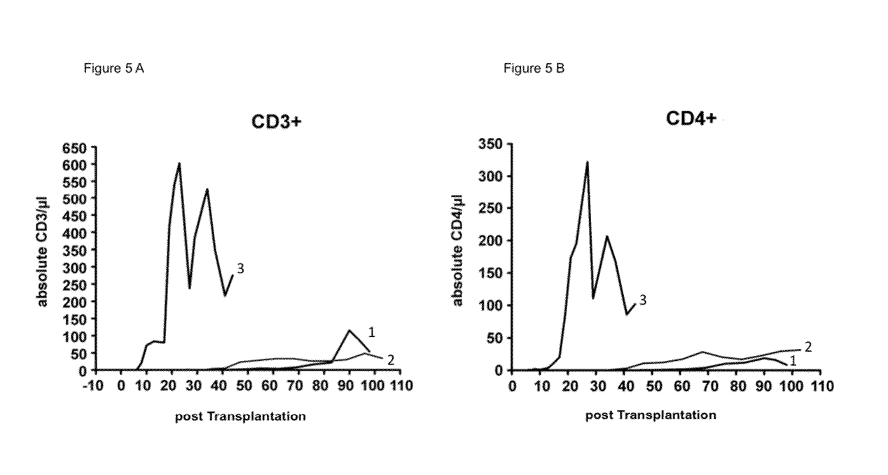

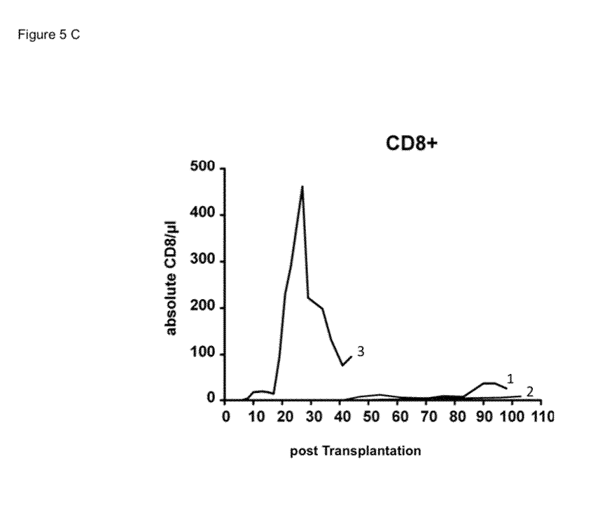

1. Transplant: Un-manipulated bone marrow of MUD donors (2008)
2. Transplant: CD3/CD19 depleted PBSC of the mother (2009)
3. Transplant: TCRα/β/CD19 depleted PB SC of the father (9 months after 2nd transplantation)

The graphs show the concentration of (from left to right) CD3 positive cells (A), CD4 positive cells (B) and CD8 positive cells (C) at different time points after transplantation of bone marrow (cells/microliter)

### Example

### Transplantation

Eleven patients were transplanted, five with the diagnosis acute lymphatic leukemia (ALL), three patients with the diagnosis acute myeloid leukemia (AML), one patient with the diagnosis agranulocytosis, one patient with the diagnosis beta-Thalassemia, one patient with the diagnosis Inborn Error (HHS). Five of the patients had already received one stem cell transplantation; three other patients had already received two or three stem cell transplantations.

In none of the patients an immune suppression after transplantation was observed.

### Example

### MUD Donor

One patient with beta-thalassemia, one patient with ALL and one patient with Inborn Error received donor material from a MUD donor with a TCR alpha/beta depleted and CD19 depleted cell preparation.

### Example

### Chronic GvHD

One patient who had received material from a haploid donor developed chronic GvHD with mild progression.

No patient who had received material from a MUD donor developed chronic GvHD.

1. A pharmaceutical composition, comprising a cell population obtainable from bone marrow or blood, wherein the cell population is depleted of TCR alpha/beta positive cells and CD19 positive cells.

2. The pharmaceutical composition of claim 1 comprising a pharmaceutically acceptable carrier.

3. A method for the production of a cell population from bone marrow or blood, comprising depleting TCR alpha/beta positive and CD19 positive cells.

4. The method of claim 3, wherein the depletion of TCR alpha/beta positive cells is performed using an antibody or an antigen-binding fragment against TCR alpha/beta.

5. The method of claim 3, wherein the depletion of CD19 positive cells is performed using an antibody or an antigen-binding fragment against CD19.

6. Use of a method of claim 3 for reconstituting a hematopoietic system of a human in connection with a stem cell or bone marrow transplantation.

7. Use of a cell population obtained from bone marrow or blood, wherein the cell population is depleted of cells that express TCR alpha/beta and of cells that express CD19, for the reconstitution of a hematopoietic system of a human in connection with a bone marrow transplantation.

8. Use of an antibody or antigen-binding fragment against TCR alpha/beta and/or of an antibody or an antigen-binding fragment against CD19 for producing a cell population that is depleted of TCR alpha/beta and CD19.

9. A kit for producing a cell population that is depleted for TCR alpha/beta and CD19, comprising an antibody or antigen-binding fragment against TCR alpha/beta and an antibody or an antigen-binding fragment against CD19.

10. Use of the kit of claim 9 for the production of a cell population that is TCR alpha/beta negative and CD19 negative.

11. Use of the pharmaceutical composition of claim 1 for the reconstitution of hematopoietic system of a human after a stem cell or bone transplantation.

12. Use of the pharmaceutical composition of claim 2 for the reconstitution of hematopoietic system of a human after a stem cell or bone transplantation.

13. Use of a method of claim 4 for reconstituting a hematopoietic system of a human in connection with a stem cell or bone marrow transplantation.


SEQUENCE LISTING (14 sequences)

SEQ ID NO: 1 - protein, 556 aa, Homo sapiens [sequence omitted]

SEQ ID NO: 2 - DNA, 1965 bp, Homo sapiens
aggcccctgcctgccccagcatcccctgcgcgaagctgggtgccccggagagtctgaccaccatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggccccagaaccagtacgggaacgtgctgtctctccccacacccacctcaggcctcggacgcgcccagcgttgggccgcaggcctggggggcactgccccgtcttatggaaacccgagcagcgacgtccaggcggatggagccttggggtcccggagcccgccgggagtgggcccagaagaagaggaaggggagggctatgaggaacctgacagtgaggaggactccgagttctatgagaacgactccaaccttgggcaggaccagctctcccaggatggcagcggctacgagaaccctgaggatgagcccctgggtcctgaggatgaagactccttctccaacgctgagtcttatgagaacgaggatgaagagctgacccagccggtcgccaggacaatggacttcctgagccctcatgggtcagcctgggaccccagccgggaagcaacctccctggggtcccagtcctatgaggatatgagaggaatcctgtatgcagccccccagctccgctccattcggggccagcctggacccaatcatgaggaagatgcagactcttatgagaacatggataatcccgatgggccagacccagcctggggaggagggggccgcatgggcacctggagcaccaggtgatcctcaggtggccagcctggatctcctcaagtccccaagattcacacctgactctgaaatctgaagacctcgagcagatgatgccaacctctggagcaatgttgcttaggatgtgtgcatgtgtgtaagtgtgtgtgtgtgtgtgtgtgtgtatacatgccagtgacacttccagtcccctttgtattccttaaataaactcaatgagctcttccaatcctaaaaaaaaaaa

SEQ ID NO: 3 - DNA, 7828 bp, Homo sapiens
accatgcccggctaatttttttttttttttttgagaaggagtctatgtgcccagcattgttctagagcacttgcaattagtggtgaacaacacggtctctactccaaggggctcacattcttgtgcagaaaacagaaatgaacaaataaacacacaagatcatttcccgtggtagtgagagctgggatgaaaataaaacagcgtggcagggaggaggcaagtgttgtgagtctggagggttcctggagaatggggcctgaggcgtgaccaccgccttcctctctggggggactgcctgccgcccccgcagacacccatggttgagtgccctccaggcccctgcctgccccagcatcccctgcgcgaagctgggtgccccggagagtctgaccaccatgccacctcctcgcctcctcttcttcctcctcttcctcacccccatggaagtcaggcccgaggaacctctagtggtgaaggtggaaggtatgtccaaagggcagaaagggaagggattgaggctggaaacttgagttgtggctgggtgtccttggctgagtaacttaccctctctgagcctccattttcttatttgtaaaattcaggaaagggttggaaggactctgccggctcctccactcccagcttttggagtcctctgctctataacctggtgtgaggagtcggggggcttggaggtcccccccacccatgcccacacctctctccctctctctccacagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggtgagggccgggctggggcaggggcaggaggagagaagggaggccaccatggacagaagaggtccgcggccacaatggagctggagagaggggctggagggattgagggcgaaactcggagctaggtgggcagactcctggggcttcgtggcttcagtatgagctgcttcctgtccctctacctctcactgtcttctctctctctgcgggtctttgtctctatttatctctgtctttgagtctctatctctctccctctcctgggtgtctctgcatttggttctgggtctcttcccaggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccagggtatggtgatgactggggagatgccgggaagcgggggtccagagacagaggggaggggaaactgaagaggtgaaaccctgaggatcaggctttccttgtcttatctctccctgtcccagacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccaggtagagtttctctcaactgggaggcatctgtgtgggggtactgggaagaagtggaagccagtcaatcttagattcccccaacccgagggctactcccagcctcaccccaaaccccaacttccacacagaacactgactccaagtctttcttttttttgacagagtctcgctctgttgcctaggctggagtgcagtggtgccatcttgtcttggctcactgcaacctccgcctcccaggttcaagtgattcccctgcctcagcctcctgagtagctgggattacaggtgcccaccaccacgcctggctaattttttttttttttttgagacggagtcttgcactgtcacccaggctggagtgcagtggcacgatctcagctcactgcaacctccaccttccaggttcaagtgattctcctgcctcagcctcccgagtagctgggattaaagcctggctaattttttttgtatttttagtagagatggggtttcattatgttggccaggctggtctcaaactcctgacctcgtgatccacccgcctcggcctcccaaagtgctgggattacagacatgagccacagggccgggccaagcctaattttgtatttttagtagagatggggtttctccctgttggaccaggctggtcttgaactcctgacttcaggtgatctgcctgccttggcctcccaaagtactgggattacaggcataagccaccgcacctggcctagacttcaagtctttcttccctcgcttccaagacactacttttctgggtcttcacctaccattgcttgcgcctgcccaccagcttgggtggagtcttccttcctccccaactcctcactcttggagccctgggccctcttcttatccctgtctgcacactttcctatttgaacttgactctcaatggcttcttgggtcaccatgccttggtgactctattccaggctccatactcagccatctcctgtgccatttgatatcccatggacacctcaggctcaacagatacaaaatcaaactcaatgtcttccccaagtatagtcttcttggtggcccagtgtaagcagagggcaccaccacctgctccctcgcccaggctaagaacctgggcatccttctttttcctcaccccgtccaacaaactggtcacagtgttctgccaattctctctccatgcaatcctatcatgctatcctaactgcaattcacaaacccaaccccaactttcactccaaacttgatccaagcaatgtgctggatcccaactgtaaccttgcaaactcaactctgcccttcactttgaccgtgactatccttaattgcagcaggaaactgatcattatgctcccctcaatccacacattgcctctgagtacagccatggtttgtccacgatttgctcaaagacactgccccatgtcctgtgccagggtctgtgacaatccctgacctcctgggacatggctccttagagagaggagagcctttctcacagcttgggactttgagtctgtgtctttttttttttcttgagacggagttttgctgtggttgcccaggctggagtgcagtgatctcggctcactgaaacctccgcctcccgggttcaaacgattctcctgcctcagcctcccaagtagctgggattacaggcacccaccaccatgcccagctaatttttttgtatttttagtagagatggggtttcaccatgttggccaggctggtctcgaactcctgacctcaggtgatccacccgcctttgcctcccaaagtgctgggattacaggcgtcaaccaccgcgcccggccgagtctgtgtcttgcctctgtgcctcagacttgcggttccttgagatctcaggattgggacgtaagatgccagcctggggtcctcgtctcatagccccttccccctagtactatggcactggctgctgaggactggtggctggaaggtctcagctgtgactttggcttatctgatcttctgcctgtgttcccttgtgggcattcttcatcttcaaagaggtgagtcatgtccccagtgggtctgtccaaaccctactccatcttccccaggataagccggctctggccagtctgacaaccatctttctttcctcccatccctcccttcaagaccccagaatcctgttctccccagtcttcctctagcctccctcaaacttcccaagcctcttgcaattttttttttttttttgagacagggtctcattctgtcaccccagctggagtgcagtggcacaatctgagctcactgtaacctctgcctcccaggcttaagtgattcttgtgcttcagcctcccgagtacctgggactacaagtgtatgccaccacacccggccaattttttatatttttagtagagacgaggtttcaccatgttggccagactggtctcgaactcttgacctcaaatgatccgcccacctcggcctcccaaagtgctgggattacaggcacgagccaccgcgcccgtccgcctcgcaatttgaactcctgtctcctttgttgaaccaagtgacctccccagcacctggccccacaaatcctcaccctgccaagcagcccctcctctgatcacgccctttaactcccaccagccctggtcctgaggaggaaaagaaagcgaatgactgaccccaccaggaggtaatgcaaccagtgcaccccgcggtaacaccctccaccttcactttatgccttgcacttactgtttcctctgcccaggggttctttgctccgtctctactgtttcaaatactgcccaacctcaaagcccagctccaaagctacctcctctgtgaagaactccttggaaatgatcatctcagactcctctattggctgtcccagcacaagtgatcacgtttaacttctgaaggcctggacagaatcttgagtgggtccgccattccattccaagtcggccctcaccgtgcacttcctcttctcccgccagattcttcaaagtgacgcctcccccaggaagcgggccccagaaccagtacgggaacgtgctgtctctccccacacccacctcaggcctcggtaagaggcaccgcccctccagcctatagctccgccccagatccggggctccacccccactctcctcatccctccaatccgctgtgcgccaagccttctggagctcggaactccgcccccggggcggggagtcccgcccagctatgagccccgcctctagaaccagaccccgcctccagggctcagagccacgcccccaggacccagagcctgaagtcgtaatcaagagcagaacttcgccccagaactgaaggcctcggccctagatttagattccgccccagggttcaaggccgggttcctagacccagagtccattcgcagagcccaaaacatcctcttcccgtgccccgccgcgcggacccttagccttgaccgcccccatctcttctgaccccgtcttacaatgcccctctcaccaggacgcgcccagcgttgggccgcaggcctggggggcactgccccgtcttatggaaacccgagcagcgacgtccaggcggatggagccttggggtcccggagcccgccgggagtgggtgaatgactgggagagggaagggtcgttccccacatggagggggttggagcggtctgtggcccgaatagtggactgggccctggaggagagggggcatgactcggttccccatccccatccccaaacccccaggcccagaagaagaggaaggggagggctatgaggaacctgacagtgaggaggactccgagttctatgagaacgactccaaccttgggcaggaccagctctcccagggtaaggctgccctcccccgtggccccccacctctgcggtggcctgtggactcccatggacacccctccttctccaccagatggcagcggctacgagaaccctgaggatgagcccctgggtcctgaggatgaagactccttctccaacggtaacttggggcctttgtgggacctcagagacttaggtgtaattgcagcgctgtgacactcctagaaggggtccctggagttctctctcttctgccacagctgagtcttatgagaacgaggatgaagagctgacccagccggtcgccaggacaatgggtgtgtgtgaggatggcaacagtccaggggggaggcggaggacacctggaggccaggaggaatagtaacctccctcttccctttccagacttcctgagccctcatgggtcagcctgggaccccagccgggaagcaacctccctgggtgagagatgctttcaatcagactgccttgcccagcttgggtgacctggcctcagctctgacaccagatccaactttgacctgaccctgaccccaaacccgaacccaatcctgtgactcctctcacctcaacactgagccccatcccccatcctgagccccatcccccatcctgacccccaatatttaccccctccctaactgtgaatatcaacaccgatcccaatgcagtatcagcctggacttgatctccacctcacctcagccccagtgcagacctcaacttggaccccagcttactctgcagcttcttcatgactctgactccgactccctccagtttcttctttttctttttcttttttttgagacggagtctccctctgttgcccaggctggagtgcagttgccacctctgcctcctaggttcaagcgattctcatgcctcagcctcctgagtagctgggattatagacgtttgccaccacacctggctaatttttgtattttcagtagagacagggtttcgccatgttggccagactggtctccaactcctggcctctagtgatctgcccgccttggcttcccaaagtgctgggattacaggcatgagccaccacgcccagcccagttctgttcttgaccccttccttagccataatctaacccatatctaaccctgaccctacagctaactggggccccaaactcaatgctaaccaaatcaccccttcccagcacagcatgggtaatgctcctcaccttcctctgcccctcagtcttcctccttaccgtaggctgtacttcccatgccctagcctccaattctccatcccccgcccaagcagggtcccagtcctatgaggatatgagaggaatcctgtatgcagccccccagctccgctccattcggggccagcctggacccaatcatgaggaaggtgggtgcttctgccgctgtcccctgctgtcccctgggctgactttgccttccagcctacttccagtgccacccatgttctcctcctccctggtcctatccagatgcagactcttatgagaacatggataatcccgatgggccagacccagcctggggaggagggggccgcatgggcacctggagcaccaggtgatcctcaggtggccaggtgagctgggactgcccctagggaaagcggggagggagggagataggcacggatggcagtggctgctggctttcagggagggagagggaacagggttcctagggcctggtgggcagggggaggactgctggacccctccccatcaccgtttcttctgcatagcctggatctcctcaagtccccaagattcacacctgactctgaaatctgaagacctcgagcagatgatgccaacctctggagcaatgttgcttaggatgtgtgcatgtgtgtaagtgtgtgtgtgtgtgtgtgtgtgtatacatgccagtgacacttccagtcccctttgtattccttaaataaactcaatgagctcttccaatcctatgaagtagtgccattgtgtgggaaggaagggaaggaaaggaaaggaaggaaagggaagggaaggaaaggaagggaaggaaaggcgt

SEQ ID NO: 4 - protein, 142 aa, Homo sapiens
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 5 - DNA, 426 bp, Homo sapiens
ccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 6 - DNA, 3430 bp, Homo sapiens
ttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagccaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaagtacgccagaaagctgttgatcggcttcaagcagggaagggacacctaatttgcttttcttttctttttttttttttttttttttttttttttgagatggagttttgctcttgttgcccaggctggagtgcaatggtgcatcttggctcactacaagcctctgcctcccaggttcaagtgattctcctgcctcagcctcccaagtagctgggattacaggcacccaccaccatgcccggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcacggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctcattggactctagaatgaagccaggcagagcagggtctatgtgtgatggcacatgtggccagggtcatgcagacatgtactttgtacaaacagtgtatattgagtaaatagaaatggtgtccaggagccgaggtatcgtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgccgaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccgtctctgactttccgttctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggcagcggaactcactaaggggcccatctggacccgaggtattgtgaagataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgcgtgcatcctaagctctgagagcaaacctccctgcagggtcttgcttttaagtccaaagcctgagcccaccaaactct

SEQ ID NO: 7 - protein, 177 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 8 - DNA, 531 bp, Homo sapiens
gaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 9 - DNA, 2042 bp, Homo sapiens
tgcatcctagggacagcatagaaaggaggggcaaagtggagagagagcaacagacactgggatggtgaccccaaaacaatgagggcctagaatgacatagttgtgcttcattacggcccattcccagggctctctctcacacacacagagcccctaccagaaccagacagctctcagagcaaccctggctccaacccctcttccctttccagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcagacaagactagatccaaaaagaaaggaaccagcgcacaccatgaaggagaattgggcacctgtggttcattcttctcccagattctcagcccaacagagccaagcagctgggtcccctttctatgtggcctgtgtaactctcatctgggtggtgccccccatccccctcagtgctgccacatgccatggattgcaaggacaatgtggctgacatctgcatggcagaagaaaggaggtgctgggctgtcagaggaagctggtctgggcctgggagtctgtgccaactgcaaatctgactttacttttaattgcctatgaaaataaggtctctcatttattttcctctccctgctttctttcagactgtggctttacctcgggtaagtaagcccttccttttcctctccctctctcatggttcttgacctagaaccaaggcatgaagaactcacagacactggagggtggagggtgggagagaccagagctacctgtgcacaggtacccacctgtccttcctccgtgccaacagtgtcctaccagcaaggggtcctgtctgccaccatcctctatgagatcctgctagggaaggccaccctgtatgctgtgctggtcagcgcccttgtgttgatggccatggtaagcaggagggcaggatggggccagcaggctggaggtgacacactgacaccaagcacccagaagtatagagtccctgccaggattggagctgggcagtagggagggaagagatttcattcaggtgcctcagaagataacttgcacctctgtaggatcacagtggaagggtcatgctgggaaggagaagctggagtcaccagaaaacccaatggatgttgtgatgagccttactatttgtgtggtcaatgggccctactactttctctcaatcctcacaactcctggctcttaataacccccaaaactttctcttctgcaggtcaagagaaaggatttctgaaggcagccctggaagtggagttaggagcttctaacccgtcatggtttcaatacacattcttcttttgccagcgcttctgaagagctgctctcacctctctgcatcccaatagatatccccctatgtgcatgcacacctgcacactcacggctgaaatctccctaacccagggggaccttagcatgcctaagtgactaaaccaataaaaatgttctggtctggcctgactctgacttgtgaatgtctggatagctccttggctgtctctgaactccctgtgactctccccattcagtcaggatagaaacaagaggtattcaaggaaaatgcagactcttcacgtaagagggatgaggggcccaccttgagatcaatagcagaa

SEQ ID NO: 10 - protein, 179 aa, Homo sapiens
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 11 - protein, 178 aa, Homo sapiens
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 12 - DNA, 537 bp, Homo sapiens
gaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 13 - DNA, 534 bp, Homo sapiens
gacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 14 - DNA, 2008 bp, Homo sapiens
atggcgtagtccccaaagaacgaggacctagtaacataattgtgcttcattatggtcctttcccggccttctctctcacacatacacagagcccctaccaggaccagacagctctcagagcaaccctagccccattacctcttccctttccagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcaggtgagtggggcctggggagatgcctggaggagattaggtgagaccagctaccagggaaaatggaaagatccaggtagcggacaagactagatccagaagaaagccagagtggacaaggtgggatgatcaaggttcacagggtcagcaaagcacggtgtgcacttcccccaccaagaagcatagaggctgaatggagcacctcaagctcattcttccttcagatcctgacaccttagagctaagctttcaagtctccctgaggaccagccatacagctcagcatctgagtggtgtgcatcccattctcttctggggtcctggtttcctaagatcatagtgaccacttcgctggcactggagcagcatgagggagacagaaccagggctatcaaaggaggctgactttgtactatctgatatgcatgtgtttgtggcctgtgagtctgtgatgtaaggctcaatgtccttacaaagcagcattctctcatccatttttcttcccctgttttctttcagactgtggcttcacctccggtaagtgagtctctcctttttctctctatctttcgccgtctctgctctcgaaccagggcatggagaatccacggacacaggggcgtgagggaggccagagccacctgtgcacaggtacctacatgctctgttcttgtcaacagagtcttaccagcaaggggtcctgtctgccaccatcctctatgagatcttgctagggaaggccaccttgtatgccgtgctggtcagtgccctcgtgctgatggccatggtaaggaggagggtgggatagggcagatgatgggggcaggggatggaacatcacacatgggcataaaggaatctcagagccagagcacagcctaatatatcctatcacctcaatgaaaccataatgaagccagactggggagaaaatgcagggaatatcacagaatgcatcatgggaggatggagacaaccagcgagccctactcaaattaggcctcagagcccgcctcccctgccctactcctgctgtgccatagcccctgaaaccctgaaaatgttctctcttccacaggtcaagagaaaggattccagaggctagctccaaaaccatcccaggtcattcttcatcctcacccaggattctcctgtacctgctcccaatctgtgttcctaaaagtgattctcactctgcttctcatctcctacttacatgaatacttctctcttttttctgtttccctgaagattgagctcccaacccccaagtacgaaataggctaaaccaataaaaaattgtgtgttgggcctggttgcatttcaggagtgtctgtggagttctgctcatcactgacctatcttctgatttagggaaagcagcattcgcttggacatctgaagtgacagccctctttctctccacccaatgctgctttctcctgttcatcctgatggaagtctcaacaca